(19) United States
(12) Patent Application Publication
(10) Pub. No.: US 2008/0142793 A1
(43) Pub. Date: Jun. 19, 2008

(54) Organic Semiconductors

(76) Inventors: Ming L. Tang, Stanford, CA (US); Zhenan Bao, Stanford, CA (US); Toshihiro Okamoto, Sunnyvale, CA (US)

Correspondence Address: CRAWFORD MAUNU PLLC, Suite 390, 1270 Northland Drive, St. Paul, MN, 55120, US

(21) Appl. No.: 11/939,312

(22) Filed: Nov. 13, 2007

Related U.S. Application Data
(60) Provisional application No. 60/858,206, filed on Nov. 10, 2006.

(51) Int. Cl. H01L 51/30; C07D 333/54

(52) U.S. Cl. 257/40; 549/42; 549/41; 257/E51.024

(57) ABSTRACT

Organic semiconducting devices and applications exhibit high performance largely due to a treatment substrate interacting with an asymmetric linear compound. According to an example, an organic compound arrangement includes a treated substrate and an asymmetric linear compound on the treated substrate. The compound includes an acene and a thiophene unit fused to the acene and exhibits high mobility and, in some applications, a correspondingly high on/off ratio. The compound arrangement is suitable for implementation with a variety of semiconducting applications, such as thin-film applications, solar applications and transistor applications.

### RELATED PATENT DOCUMENTS

This patent document claims the benefit, under 35 U.S.C. § 119(e), of U.S. Provisional Patent Application No. 60/858,206 filed on Nov. 10, 2006 and entitled “High Performance Organic Semiconductors,” which is fully incorporated herein by reference.

### FIELD OF THE INVENTION

The present invention relates generally to high performance organic semiconductors and to their uses and manufacture.

### BACKGROUND

Organic semiconductors continue to show promise for a variety of applications, and have been the subject of consideration for use in many low-cost, large area electronic applications. As an example, organic thin-film transistors (OTFTs) are a relatively viable alternative to more traditional, mainstream thin-film transistors (TFTs) based on inorganic materials. However, organic semiconductor layers often exhibit relatively low mobility and, correspondingly, relatively low performance characteristics in comparison to field-effect transistors using single-crystalline inorganic semiconductors such as Silicon and Germanium and exhibiting much higher charge carrier mobilities (μ). Consequently, electronic applications requiring very high switching speeds have not typically used OTFTs.

Prior efforts to increase semiconductor mobilities and improve other processing characteristics have involved the use of pentacene as active material in p-type organic transistors. Pentacene has demonstrated relatively high mobilities (e.g., over 1.0 cm²/Vs) and desirable on-off ratios which is an issue relevant to switching speeds. However, pentacene is subject to rapid degradation in ambient conditions, which may result from the formation of transannular endoperoxide or dimeric Diels-Alder adducts. Pentacene derivatives have also been used, but synthesis of these derivatives has often resulted in undesirable film disorder. As such, pentacene and pentacene derivatives have experienced limited application.

These and other issues have been challenging to the design, manufacture and implementation of semiconductor devices, and in particular, for those semiconductor devices employing organic semiconductor materials.

### SUMMARY

The present invention is directed to the above-mentioned challenges and others related to the types of applications discussed above and in other applications. These and other aspects of the present invention are exemplified in a number of illustrated implementations and applications, some of which are shown in the figures and characterized in the claims section that follows.

In certain embodiments, the present invention relates to asymmetric linear compounds that facilitate desirable mobility and related performance in semiconductor applications. Examples of such compounds include asymmetric linear acenes, such as pentacene and tetracene derivatives, with a fused thiophene unit at an end of the linear acene. These compounds are applicable for implementation with substrate materials that, when employed with the compounds, facilitate desirable mobilities and on-off ratios for semiconductor applications.

According to one aspect, the present invention is directed to organic thin-film semi-conducting components and devices such as an organic thin-film transistor (OTFT), and devices built using such components, where one of primary molecules for the active material in the organic thin-film semi-conducting component is based on one of the above-discussed compounds.

According to other aspects, the present invention is directed to the partial and/or complete manufacture of such organic thin-film semi-conducting components and devices, where one of primary molecules for the active material in the organic thin-film semi-conducting component is based on one of the above-discussed compounds.

According to other aspects, the present invention is directed to the use of such devices, and to the use of such components and devices employing such related derivatives in combination with other materials including inorganic semiconductors, such as Silicon and Germanium.

According to another example embodiment of the present invention, a semiconducting organic compound arrangement includes a substrate, a film on the substrate and an asymmetric linear compound on the film. The compound includes an acene and a thiophene unit fused to the acene and is configured and arranged to exhibit a high mobility (e.g., at least about 0.55-0.7 cm²/Vs).

According to another example embodiment of the present invention, an organic thin-film semi-conducting device includes a substrate, a film on the substrate, an asymmetric linear compound and a gate. The compound includes a fused thiophene unit which is on the film and connects electrodes for passing current. The gate switches the asymmetric linear compound for conducting current between the electrodes, and the compound is responsive to the gate by exhibiting an on-off ratio of at least about 10⁶.

Another example embodiment is directed to an organic thin-film semiconductor device. The device includes a substrate with an octadecyltrichlorosilane (OTS) film on the substrate, and an array of organic thin-film transistors. Each transistor includes a source, a drain, a channel region electrically connecting the source and drain and an asymmetric linear compound on the OTS film. The compound includes an acene and a thiophene unit fused to the acene. A gate electrode capacitively couples to the channel region to switch the region for selectively passing current between the source and drain. A controller controls the gate electrodes and the operation of the transistors.

For use as a channel region in a semiconductor arrangement, another example embodiment is directed to a semiconducting organic compound arrangement comprising an asymmetric linear compound including an acene and a thiophene unit fused to the acene. In some applications, the organic compound arrangement exhibits a mobility of at least about 0.55 cm²/Vs. In other applications, the organic compound arrangement exhibits a mobility of less than about 0.55 cm²/Vs and may, for example, be implemented with a mobility-enhancing material such as an OTS film as described above.

According to another example embodiment, a semiconducting organic compound arrangement includes an asymmetric linear compound including an acene and a thiophene unit fused to the acene.

In connection with another example embodiment of the present invention, a semiconductor material includes a tetraceno[2,3-b]thiophene ring functionalized at 7 and 10 positions (e.g., with examples described and shown in the following discussion).

The above summary is not intended to describe each illustrated embodiment or every implementation of the present invention.

### BRIEF DESCRIPTION OF THE DRAWINGS

The invention may be more completely understood in consideration of the detailed description of various embodiments of the invention that follows in connection with the accompanying drawings in which:

FIGS. 1A and 1B show an approach to the manufacture of an organic semiconductor device, according to an example embodiment of the present invention, in which

FIG. 1A shows example synthesis of organic compounds, and

FIG. 1B shows application of such compounds in a semiconductor arrangement;

FIG. 2 shows transfer curves of example compounds as implemented in connection with other example embodiments of the present invention; and

FIG. 3 shows a top-contact transistor arrangement that is based upon a linear organic compound, according to another example embodiment of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

The present invention is directed to high performance organic semiconductors and to their uses and manufacture, as well as related types of materials, devices, methods of manufacture and approaches. These and other aspects of the present invention are exemplified in a number of illustrated implementations and applications, some of which are shown and characterized in the following description, the figures, and the claims section that follows.

According to an example embodiment of the present invention, an organic thin-film device includes a linear compound including an acene with a fused thiophene unit at an end of the acene. The compound is arranged on a substrate material, such as a bulk or layered substrate, that facilitates desirable mobility characteristics. In some applications, compound mobility exceeds about 0.55 cm²/Vs, and in other applications, exceeds about 1.0 cm²/Vs. For certain implementations, the compound is arranged to exhibit a significantly high on-off ratio, when implemented with a semiconducting device (such as a transistor), on the order of about 10⁶ or higher. These approaches are useful, for example, in organic thin-film transistors in applications directed to large-area coverage, structural flexibility, low-temperature processing and/or low cost. Such thin-film-transistor applications include switching devices for active-matrix flat-panel displays, smart cards and electronic identification tags, to name a few.

In connection with various example embodiments, it has been discovered that material layer or surface treatment-type material can be used to enhance the on/off ratio of organic semiconductors employing acenes with fused thiophene units. For instance, it has been discovered that an octadecyltrichlorosilane (OTS) film coated onto a Silicon-based substrate facilitates an increased on/off ratio of asymmetric linear acenes containing thiophene, such as an acene with 1-5 linear benzene rings and a thiophene unit fused to one of the benzene rings at an end of the acene. This combination of substrate material/treatment with a linear acene is useful for many semiconductor applications (as discussed above), with asymmetric acene/thiophene compounds switched between on and off states for selectively passing current.

In connection with further example embodiments, it has also been discovered that such compounds exhibit desirable stacking (and related) characteristics when coupled with a substituent having a length that is about half the length of the linear acene. For instance, when a spherical substituent having about half the length of a pentacene-based linear acene is coupled to (or included with) the compound, a two-dimensional (2-D) bricklayer-type film structure with packing in a tight brick-like stack is obtained. Similar approaches involve the creation of a 2-D π-stack structure with a tetracene-based linear acene. These approaches facilitate desirable film stacking and mobility.

According to various example embodiments, a semiconducting organic compound arrangement includes a substrate, a film on the substrate and an asymmetric linear compound on the film. The compound includes an acene and a thiophene unit fused to the acene and exhibiting high mobility. For instance, while other approaches have involved similar compound arrangements exhibiting mobilities of up to about 0.5 cm²/Vs, the present invention is directed to embodiments involving mobilities exceeding these. In addition, while other approaches have been limited in application to certain compounds, various aspects of the present invention are directed to the use of compounds at lower mobilities (e.g., less than about 0.55 cm² Vs), yet facilitating desirable operational characteristics, such as enhanced mobility or on/off ratio (e.g., using an OTS film). For instance, some applications involve compounds exhibiting mobilities of about 0.8 cm²/Vs, and other applications involve compounds exhibiting mobilities up to and/or exceeding about 1.2 cm²/Vs.

In certain embodiments, the present invention relates to asymmetric linear pentacene and tetracene derivatives containing fused thiophene units. A first compound, tetraceno[2,3-b]thiophene (hereinafter referred to as compound 1), is a reddish purple compound with a conjugation length between tetracene and pentacene. A second compound, anthra[2,3-b]thiophene (compound 2), is a bright yellow molecule with a conjugation length between tetracene and anthracene. These compounds (or similar compounds as appropriate) are manufactured and implemented in a variety of manners.

In one embodiment, semiconductor devices are formed by evaporating thin films of compound 1 and/or compound 2 on a substrate such as SiO₂/Si, treated (i.e., layered) with octadecyltrichlorosilane (OTS), to make organic thin film transistors (OTFTs) with top-contact geometry. The average mobility of compound 1 is about 0.31 cm²/Vs at 60° C. (e.g., and up to a maximum of about 0.47 cm²/Vs), and the average mobility of compound 2 is more than about 0.1 cm²/Vs. These evaporated films (or portions thereof) are used to selectively connect circuit nodes in response to a bias applied to the channel such as by a transistor gate. For both compounds 1 and 2, the on/off ratio on the order of 10⁶ for the OTS treated surface.

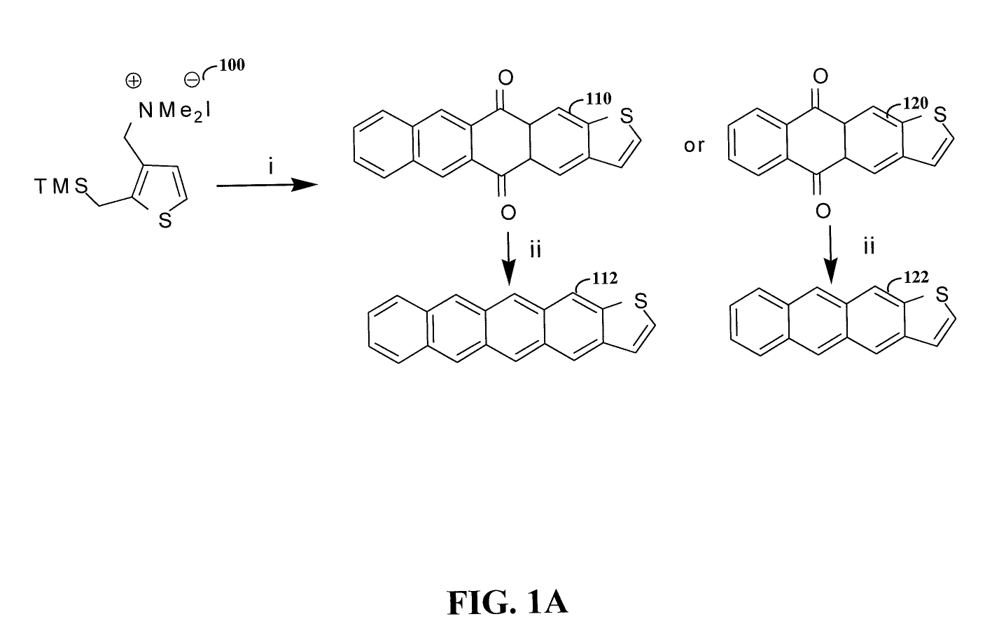

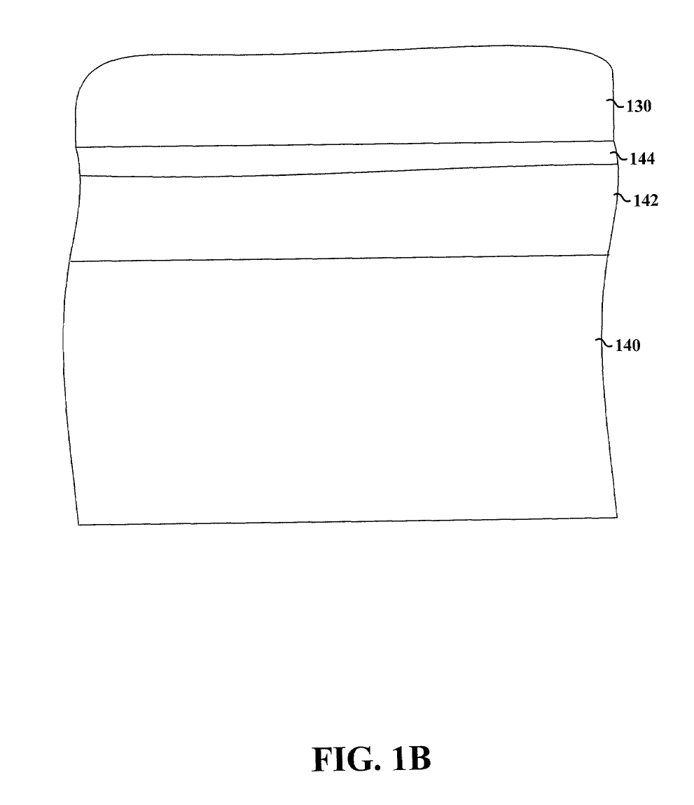

FIGS. 1A and 1B show an approach to the manufacture of such an arrangement, according to another example embodiment of the present invention. In FIG. 1A, the synthesis of each of compound 1 (112) and compound 2 (122) are shown involving two steps (i and ii). A known compound 100 (2-(trimethylsilylmethyl)-3-(trimethylammonium)-thiophene iodide), is used in the first step to create quinone precursors 110 and 120 respectively for each of the compounds 112 and 122. The quinone precursors are then reduced in the second step to compounds 1 and 2 as shown.

In FIG. 1B, an organic semiconductor arrangement has been formed using one or the compounds 1 and 2. A material layer 130 including one of the compounds 1 and 2 is formed on a substrate arrangement 140 having a silicon substrate 142, a SiO₂ layer 144 on the silicon substrate and an OTS film 146 on the SiO2 layer. Depending upon the application, other substrate treatments such as phenylsilane are used in lieu of or in addition to the OTS film treatment.

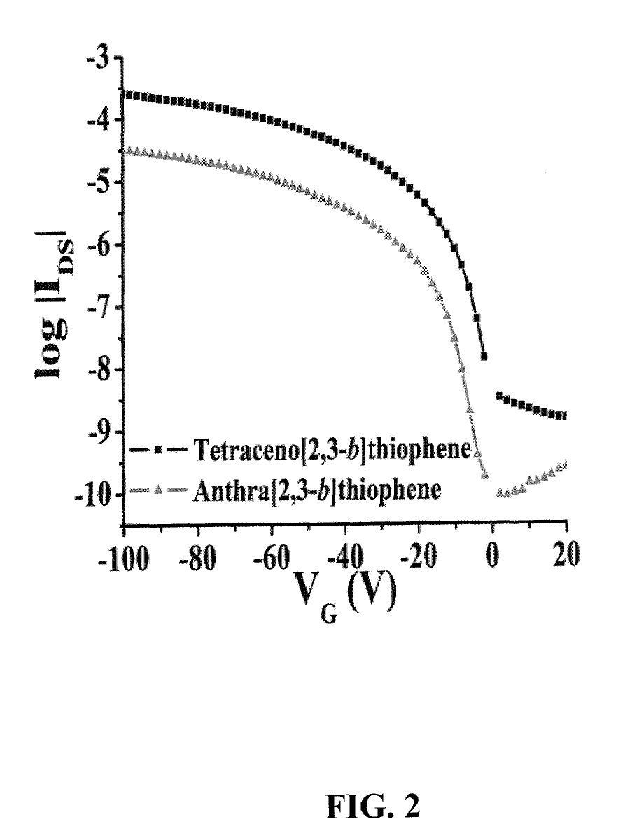

FIG. 2 shows transfer curves of compounds 1 and 2 implemented on an OTS-treated SiO₂/Si substrate, such as that shown in FIG. 1B, according to another example embodiment of the present invention. The compounds 1 and 2 are respectively operated at a temperature 60° C. and room temperature.

The substrate is formed to different set thicknesses depending upon the applications, and as discovered in connection with a particular embodiment, relatively thicker films on SiO₂/Si exhibit higher mobility (e.g., 1220 nm films exhibit a mobility of 0.006 cm2/Vs, versus 1×10⁻⁵ cm2/Vs for 40 nm films). With relatively thicker films, the grains of the film are readily connected with an immediately adjacent dielectric at the interface therebetween.

Other example embodiments are directed to the use of a linear compound as discussed herein, with additional substituted molecules selected to facilitate packing in thin film applications. Example groups that are used for substitution in different applications include triisopropyl-, triethyl- and trimethyl-silylethynyl groups. For instance, a 5,12-bis(triisopropylsilylethynyl)tetraceno[2,3-b]thiophene has been discovered to exhibit a mobility of about 1.25 cm²/Vs, while 5,10-bis(triethylsilylethynyl)anthra[2,3-b]thiophene and 5,10-bis(trimethylsilylethynyl)anthra[2,3-b]thiophene has been discovered to exhibit a mobility of 10⁻⁴ cm²/Vs, respectively formed as films on phenylsilane and octadecyltrichlorosilane-treated (e.g., coated) surfaces. Molecules including bis(trialkylsilylethynyl)tetraceno[2,3-b]thiophene-type and anthra[2,3-b]thiophene-type molecules are used in many applications. Other molecules that are used in applications such as in organic thin-film transistors (OTFTs) include 6,13-bis(triisopropylsilylethynyl)pentacene, 2,6-Bis[2-(4-pentylphenyl)vinyl]anthracene, functionalized anthradithiophene molecules which have solubilizing groups attached, and 6,13-bis(4-pentylphenylethynyl)pentacene, which exhibits a mobility of about 0.52 cm²/Vs when drop-cast from chlorobenzene solution. All these molecules have hole mobilities>0.1 cm²/Vs, are soluble in common organic solvents, and are generally stable under ambient conditions. These and other molecules may be implemented in connection with linear compounds, such as the linear acene compounds with fused thiophene units as discussed above, with application to substrates such as OTS-treated SiO₂ substrates.

Close packing of these linear compounds with substituted molecules in film form is effected in different manners for various example embodiments. The size of the substituent is set to about half the length of linear acenes to facilitate a compact structure (e.g., a 2-D bricklayer structure). For example, a triisopropylsilyl (TIPS) substituent is used with a pentacene-based compound in certain embodiments to provide functionalized pentacene with thin film mobilities as high as about 0.4 cm²/Vs when vacuum-deposited, and 1.8 cm²/Vs when drop-cast from solution, and desirable stacking. TESethynyl substituted anthradithiophene is grown in a close-packed 2-D π-stack structure for various embodiments, and facilitates a drop-cast mobility of about 1.0 cm²/Vs. In general, the size of the substituent is set, relative to the linear compound and/or the linear acene therein to mitigate or avoid undesirable stacking, such as 1-D π-stack packing that can result when relatively small substituents are used, or a herringbone structure that can result when relatively large substituents are used.

In other embodiments, stacking characteristics are used together with additional characteristics, such as grain structure, nucleation, growth and surface selection, which are controlled to achieve desirable mobility for substituted linear compounds. For instance, one application is directed to the use of TIPS substitutents with a tetraceno[2,3-b]thiophene-based compound, which facilitates 2-D π-stacking. Where implemented in thin-film transistors (TFTs), TIPSEthiotet, TIPSethynyl tetraceno[2,3-b]thiophene channel regions are used to achieve desirable mobilities as high as or higher than 1.25 cm²/Vs (e.g., when deposited as a thin film under high vacuum). In addition, anthra[2,3-b]thiophene series compounds are implemented with TES and trimethylsilyl (TMS) ethynyl substituents to facilitate a mobility of about 10⁻⁴ cm²/Vs on silane-treated surfaces. These and other arrangements exemplify substitutent-based approaches that are applicable for use in connection with various example embodiments.

Other compounds used in connection with various example embodiments include functionalization at the 7 and 10 positions on a tetraceno[2,3-b]thiophene ring, denoted by R₇ and R₈ in the molecule below.

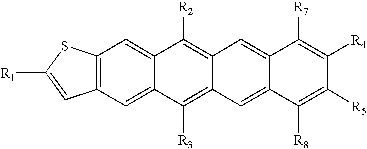

Other embodiments are directed to the implementation of compounds as follows:

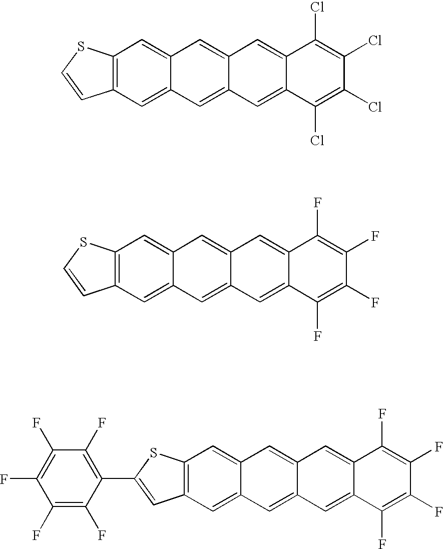

In addition to the above, various derivative compounds may be used without departing from the scope of certain aspects of the disclosure. Such derivatives of an asymmetric linear compound (of pentacene or tetracene) can be categorized, for example, as Small Molecules or Polymers. Small Molecules are exemplified as follows:

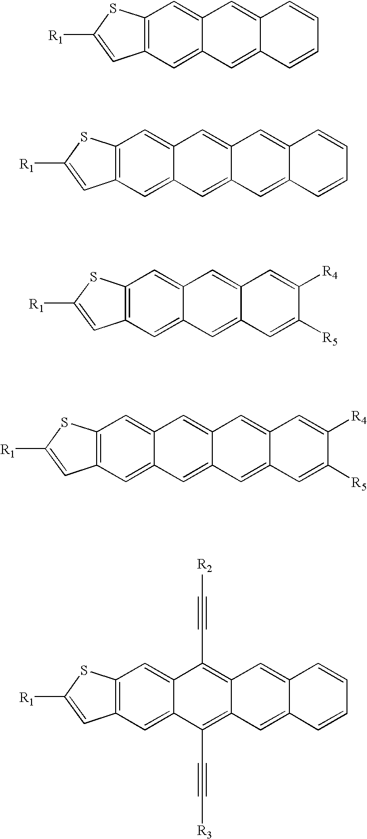

where R₁=I, Br, Cl, F, alkyl, perfluoroalkyl, carbonyl, ≡—R

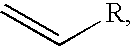

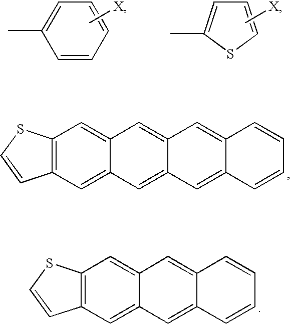

where R₂, R₃=triisopropylsilyl (TMS), n-alkyl, t-butoxy, isopropyl, and where R₄, R₅=R₁, R₂. A Polymer is exemplified as:

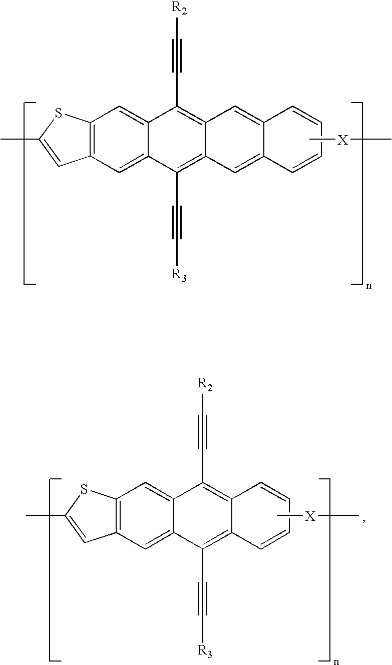

where X=aromatic units such as thiophene or phenyl based units, or other co-monomer groups.

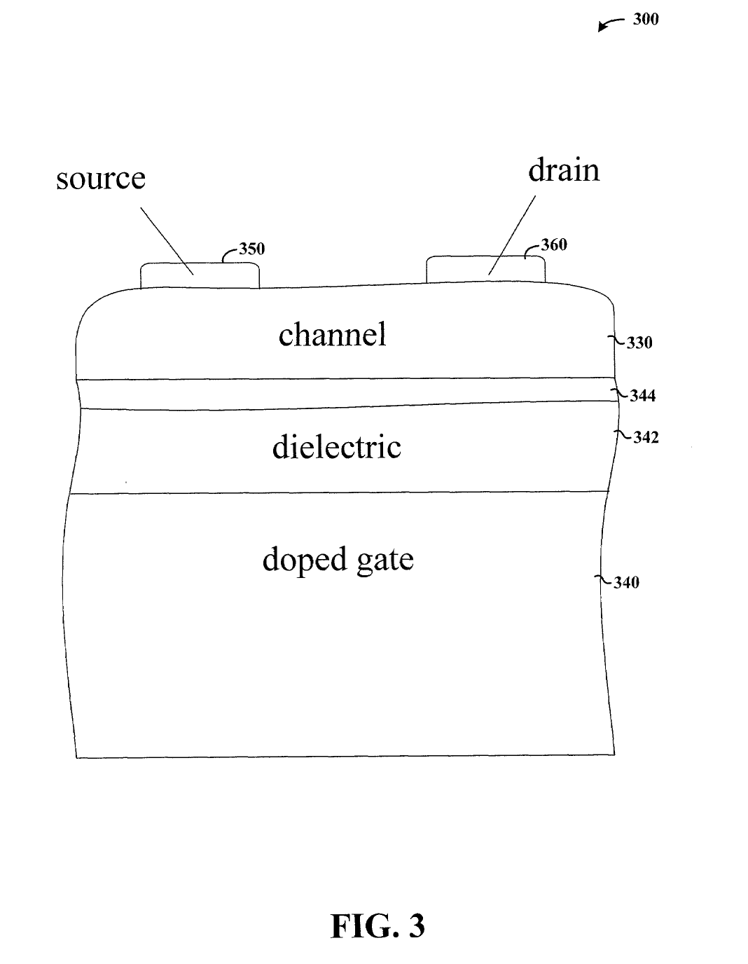

FIG. 3 shows a top-contact transistor arrangement 300 that is based upon a linear organic compound, according to another example embodiment. The arrangement 300 includes a substrate 340 doped for implementation as a gate structure, with a dielectric layer 342 on the doped substrate. A treatment layer 344 is formed on the dielectric layer 342 and includes, for example, a material such as OTS that facilitates desirable on-off characteristics. A channel layer 330 includes a linear compound as described herein, such as one of the compounds 1 and 2 or one of the substituted compounds discussed above. A source 350 and drain 360 are formed on the channel layer 330, which electrically connects the source and drain in response to a bias applied by the doped gate 340, via the dielectric 342 and treatment layer 344.

The arrangement 300 is formed using one or more of a variety of processes. In one application, the substrate 340 is n-doped and the dielectric layer 342 is a dry, thermally grown 300 nm silicon dioxide layer with a capacitance per unit area of about 1.0×10⁻⁸ F/cm². The treatment layer 344 is an OTS film coated onto the dielectric layer 342, and an organic semiconductor layer 330 of a linear compound is evaporated on the treatment layer at a rate of about 0.2-0.3 Å/s under a pressure of about 4-6×10⁻⁶ mmHg to a final thickness of about 45 nm. Shadow masks (e.g., with W/L of 20 (W=1000 μm, L=50 μm)) are used after the evaporation of the organic semiconductor to deposit gold source and drain electrodes 350 and 360.

While FIG. 3 shows one example transistor approach with application to standalone and integrated devices with a multitude of such transistors, the compounds, substituted compounds and related structures as described herein are applicable for use with many semiconductor devices. Devices such as solar cells, photovoltaic devices, thin-film transistors and others are readily manufactured and implemented using these approaches.

The various molecules and compounds described are manufactured using a variety of approaches. For example, in some applications, TESacetylene is manufactured using hexane solvents, and TMSacetylene is manufactured using ether as a solvent to generate acetylide, with yields of >70% obtained. These molecules are then purified by consecutive recrystallizations in degassed hexanes. For TIPSEthiotet, films formed at a substrate temperature of about 60° C. exhibit relatively smooth surfaces and 2D-type of growth, facilitating desirable mobility (e.g., an average of 0.80 cm²/Vs and 0.78 cm²/Vs on bare SiO₂ and OTS-treated SiO₂). Consistent with many applications, at smaller values of full-width-at-half-maximum (FWHM) and the higher peak-intensities, mobility is higher.

As consistent with the discussion herein, the present invention is believed to be useful for a variety of different thin-film organic applications, and the invention has been found to be particularly suited for such applications improving the reliability and predictability of the base (active) material in thin-film organic components and devices. For example, in addition to mainstream electronic OTFT-based applications, applications for the present invention include (large) displays, solar cells, and solar-cell arrays.

Various aspects of the invention may also be appreciated through a discussion example compounds and their implementation, including the discussion in the documents that form part of the above-identified provisional patent application and identified as Appendices A and B therein. The references disclosed in these Appendices A and B provide background information for the present invention and for the detailed embodiments disclosed in the Appendices A and B; and discuss applications which would benefit from the present invention. These Appendices and their cited references are fully incorporated herein by reference.

The various embodiments described above are provided by way of illustration only and should not be construed to limit the invention. Based upon the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention, including that set forth in the following claims.

What is claimed is:

1. A semiconducting organic compound arrangement comprising
a substrate;
a film on the substrate;
on the film, an asymmetric linear compound including an acene and a thiophene unit fused to the acene and being configured and arranged to exhibit a mobility of at least about 0.55 cm²/Vs.

2. The arrangement of claim 1, wherein the substrate, the film and the compound are configured and arranged as part of a semiconducting device that exhibits an on/off ratio on the order of about 10⁶.

3. The arrangement of claim 1, wherein the film is an octadecyltrichlorosilane (OTS) film that facilitates the exhibited mobility.

4. The arrangement of claim 1, wherein the asymmetric linear compound consists of
an acene including a number N of linearly-arranged fused benzene rings, N being an integer between about 1 and 5, and
a thiophene unit fused to a benzene ring at an end of the acene.

5. The arrangement of claim 1, wherein the asymmetric linear compound consists of the acene and the thiophene unit.

6. The arrangement of claim 1, wherein the asymmetric linear compound further includes a spherical substituent that is about half the length of the acene.

7. The arrangement of claim 1, wherein
the asymmetric linear compound further includes a spherical substituent that is about half the length of the acene, and
the asymmetric linear compound is in a layer of said asymmetric linear compounds in a two-dimensional stacked structure.

8. An organic thin-film semi-conducting device, comprising:
a substrate;
a film on the substrate;
an asymmetric linear compound on the film, connecting electrodes and including a fused thiophene unit; and
a gate to switch the asymmetric linear compound for conducting current between the electrodes, wherein the compound is responsive to the gate by exhibiting an on-off ratio of at least about 10⁶.

9. The device of claim 8, wherein the asymmetric linear compound includes an acene with a thiophene unit fused to an end of the acene, and exhibits a mobility of at least about 0.7 cm²/Vs.

10. The device of claim 8, wherein the asymmetric linear compound is an acene having a number N of linearly-arranged fused benzene rings, N being an integer between about 1 and 5, with the thiophene unit fused to one of the benzene rings at an end of the acene.

11. The device of claim 8, wherein the asymmetric linear compound includes a linear derivative of tetracene, including a fused thiophene unit.

12. The device of claim 8, wherein the asymmetric linear compound includes a linear derivative of tetraceno[2,3-b]thiophene.

13. The device of claim 8, wherein the asymmetric linear compound includes a linear derivative of pentacene, including a fused thiophene unit.

14. The device of claim 8, wherein the asymmetric linear compound includes a linear derivative of anthra[2,3-b]thiophene.

15. The device of claim 8, wherein the film includes an octadecyltrichlorosilane (OTS) film that facilitates the on/off ratio.

16. The device of claim 8, wherein
the asymmetric linear compound further includes a spherical substituent that is about half the length of the acene, and
the asymmetric linear compound is in a layer of said asymmetric linear compounds in a two-dimensional stacked structure.

17. The device of claim 8, wherein
the asymmetric linear compound further includes a spherical substituent that is about half the length of the acene, and
the asymmetric linear compound is a layer of asymmetric linear compounds having a fused thiophene unit in a two-dimensional π-stacked structure.

18. An organic thin-film semiconductor device comprising:
a substrate;
an octadecyltrichlorosilane (OTS) film on the substrate; and
an array of organic thin-film transistors, each transistor including
a source,
a drain,
a channel region electrically connecting the source and drain and including an asymmetric linear compound on the OTS film, the compound including an acene and a thiophene unit fused to the acene, and
a gate electrode capacitively coupled to the channel region to switch the region for selectively passing current between the source and drain.

19. The device of claim 18, wherein for each transistor, the asymmetric linear compound exhibits an on/off ratio on the order of about 10⁶ in response to a bias applied by the gate electrode.

20. The device of claim 18, the asymmetric linear compound further includes a spherical substituent that is about half the length of the acene.

21. For use as a channel region in a semiconductor arrangement exhibiting a mobility of at least about 0.55 cm²/Vs, a semiconducting organic compound arrangement comprising an asymmetric linear compound including an acene and a thiophene unit fused to the acene.

22. The arrangement of claim 21, wherein the acene includes a number N of linearly-arranged fused benzene rings, N being an integer between about 1 and 5, and the thiophene unit is fused to a benzene ring at an end of the acene.

23. The arrangement of claim 21, wherein the semiconducting organic compound arrangement includes an octadecyltrichlorosilane (OTS) film adjacent the asymmetric linear compound and that facilitates said mobility.

24. The arrangement of claim 21, wherein the semiconducting organic compound arrangement includes a spherical substituent that is about half the length of the acene to facilitate stacking of the semiconducting organic compounds.

25. A semiconducting organic compound arrangement comprising an asymmetric linear compound including an acene and a thiophene unit fused to the acene.
